(19) United States
(12) Patent Application Publication
(10) Pub. No.: US 2020/0038587 A1
(43) Pub. Date: Feb. 6, 2020

(54) SYSTEMS AND METHODS FOR MONITORING AND CONTROLLING A STATE OF A SUBJECT DURING VOLUME RESUSCITATION

(71) Applicant: The General Hospital Corporation, Boston, MA (US)

(72) Inventors: Jin-Oh Hahn, Boston, MA (US); Andrew T. Reisner, Boston, MA (US)

(21) Appl. No.: 16/484,800

(22) PCT Filed: Feb. 12, 2018
(86) PCT No.: PCT/US18/17832

§ 371 Date: Aug. 8, 2019

Related U.S. Application Data
(60) Provisional application No. 62/457,241, filed on Feb. 10, 2017.

(51) Int. Cl. A61M 5/172; A61B 5/0205; A61B 5/1455; A61M 5/142; G16H 40/63; G16H 15/00

(52) U.S. Cl. CPC A61M 5/1723; A61B 5/0205; A61B 5/14551; A61B 5/0215; G16H 40/63; G16H 15/00; A61M 5/142

(57) ABSTRACT

Systems and methods for monitoring and/or controlling a state of a subject during volume resuscitation are disclosed. One method includes acquiring cardiovascular data from a subject, obtaining previous volume administration information, and analyzing the cardiovascular data to determine a past time trajectory for at least one cardiovascular parameter. The method also includes determining a future state of the subject with and without additional volume administration using a statistical model, and determining, based on the future state of the subject, a likelihood that the at least one cardiovascular parameter will exceed or fall below a threshold at one or more pre-determined time points. The method further includes generating a report including a recommendation for administering an additional volume to the subject based on the determined likelihood and controlling administration of an additional fluid volume to the subject based on the report.

### CROSS-REFERENCE TO RELATED APPLICATIONS

This application is based on, claims priority to, and incorporates herein by reference in its entirety U.S. Provisional Application Ser. No. 62/457,241, filed Feb. 10, 2017, and entitled “Predictive Model for Automated Decision-Support during Intravascular Volume Resuscitation.”

### BACKGROUND OF THE DISCLOSURE

The field of the invention relates to medical monitoring and intervention. More particularly, the invention relates to systems, devices, and methods for monitoring and controlling a state of a subject, such as a cardiovascular (CV) state or a circulatory state, during volume resuscitation.

When a patient loses blood volume due to hemorrhage, such as after a major traumatic injury, when there is a bleed within the gastrointestinal tract, or during surgery, the result may be insufficient blood volume for circulation. Insufficient blood volume can eventually evolve into fatal circulatory collapse, which is typically manifested as blood pressure (BP) that first falls below normal levels, then becomes dangerously low, and finally becomes unmeasurably low.

To counteract active blood loss, intravascular volume resuscitation is generally administered. Intravascular volume resuscitation involves intravenous infusions of fluids intended to increase the patient's circulating blood volume. Fluid volumes, including crystalloid fluids (such as saline or lactated Ringer's solution), colloids (such as albumen-containing solutions), and/or blood products (such as units of red cells, plasma and platelets), may be infused to increase the patient's circulating blood volume.

In general, a clinician should transfuse enough fluid to avoid dangerously low circulation. However, there are several factors that make this challenging in practice. First, the amount of blood being lost at any given time is not easily measured and, moreover, patterns of blood loss may be irregular through time, such that patients may be stable for temporary intervals, while rapidly losing large amounts of blood at other intervals. Second, there are risks to providing excessive blood transfusion, which include risks from the blood product itself (such as infection risk and deleterious pro-inflammatory effects) as well as risks of excessively high BP during active hemorrhage, which can escalate hemorrhage rate and/or promote abnormal clotting function.

Because of these challenges, clinicians must administer volume judiciously. That is, clinicians must administer enough volume so that the patient does not develop dangerously low blood volumes, but also avoid excessive volume resuscitation. Judicious administration of volume by a clinician requires significant cognitive work and attention as time progresses and the patient's state continually evolves. Furthermore, such work by the clinician is generally more reactive than proactive (for example, the clinician generally reacts to monitored changes in BP). However, clinicians may make an error of omission or commission and patients may suffer needless episodes of hypotensive hypovolemia (when insufficient volume is administered), or suffer the dangers of needless volume resuscitation (when excessive volume is administered).

In light of the above, there is a need for improved systems and methods to accurately monitor and control CV conditions of a patient during intravascular volume resuscitation.

### SUMMARY OF THE DISCLOSURE

The present disclosure overcomes the aforementioned drawbacks by providing systems and methods for monitoring and controlling a state of a subject during intravascular volume resuscitation or other volume administration applications. Specifically, a novel approach for determining a current and future state of the subject is described, using a first parameter, previous volume administration information and/or other parameters. Determinations of current and future states may then be utilized to inform additional volume administration.

In one aspect of the present disclosure, a system for monitoring a state of a subject is provided. The system includes a sensor, a processor, and an output. The sensor is configured to acquire cardiovascular data from the subject, and the processor is configured to obtain previous volume administration information, analyze the cardiovascular data to determine a past time trajectory for at least one cardiovascular parameter, and determine, using the past time trajectory and the previous volume administration information, a future state of the subject with and without additional volume administration. The processor is further configured to determine, based on the future state of the subject, a likelihood that the at least one cardiovascular parameter will exceed or fall below a threshold at one or more pre-determined time points and generate a report indicative of the future state of the subject. The output is configured to display the report to a user.

In another aspect of the present disclosure, a system for controlling a state of a subject is provided. The system includes a treatment unit and a processor. The treatment unit is configured to administer a fluid volume to the subject, and the processor is configured to receive cardiovascular data acquired from the subject, obtain previous volume administration information, and analyze the cardiovascular data to determine a past time trajectory for at least one cardiovascular parameter. The processor is also configured to determine, using the past time trajectory and the previous volume administration information, a future state of the subject with and without additional volume administration and determine, based on the future state of the subject, a likelihood that the at least one cardiovascular parameter will exceed or fall below a threshold at one or more pre-determined time points. The processor is further configured to generate a report including a recommendation for administering an additional volume to the subject based on the determined likelihood and control the treatment unit to administer the fluid volume to the subject based on the report.

In yet another aspect of the disclosure, a method for monitoring a state of a subject is provided. The method includes acquiring cardiovascular data from a subject, obtaining previous volume administration information, and analyzing the cardiovascular data to determine a past time trajectory for at least one cardiovascular parameter. The method also includes determining a future state of the subject with and without additional volume administration using a statistical model with the cardiovascular data and the previous volume administration information as inputs to the statistical model. The method farther includes determining, based on the future state of the subject, a likelihood that the at least one cardiovascular parameter will exceed or fall below a threshold at one or more pre-determined time points, generating a report indicative of the future state of the subject, and displaying the report to a user.

In yet another aspect of the disclosure, a method for controlling a state of a subject is provided. The method includes acquiring cardiovascular data from a subject, obtaining previous volume administration information, and analyzing the cardiovascular data to determine a past time trajectory for at least one cardiovascular parameter. The method also includes determining a future state of the subject with and without additional volume administration using a statistical model with the cardiovascular data and the previous volume administration information as inputs to the statistical model, and determining, based on the future state of the subject, a likelihood that the at least one cardiovascular parameter will exceed or fall below a threshold at one or more pre-determined time points. The method further includes generating a report including a recommendation for administering an additional volume to the subject based on the determined likelihood and controlling administration of an additional fluid volume to the subject based on the report.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram of an example monitoring and control system in accordance with aspects of the present disclosure.

FIG. 2 is a flowchart setting forth steps of a process in accordance with aspects of the present disclosure.

FIG. 3 is a graphical illustration showing an example of determined past and predicted future time trajectories for blood pressure in accordance with aspects of the present disclosure.

FIG. 4 is a schematic diagram describing an autoregressive exogenous model in accordance with aspects of the present disclosure.

FIG. 5 is another graphical illustration showing an example of determined past and predicted future time trajectories for blood pressure in accordance with aspects of the present disclosure.

### DETAILED DESCRIPTION

The present disclosure describes a novel approach for monitoring and/or controlling a cardiovascular (CV) state of a subject during intravascular volume resuscitation or another volume administration application. In some aspects, the provided systems and methods are directed to monitoring and determining a present and predicted future CV state of a subject in order to provide automated decision support and/or optimize administration of intravascular volume by a clinician or a system suitable to do so. For instance, a future estimated time trajectory for at least one CV parameter, such as blood pressure (BP), may be determined to estimate a likelihood for exceeding or falling below a threshold at one or more pre-determined time points. As will be appreciated from descriptions below, this allows determination for the necessity and/or timing of additional volume administration. This approach can result in proactive control with minimized effort and fewer interruptions in clinical settings, where volume administration is typically controlled by a human clinician in a reactive manner.

Used herein, the terms “volume administered” or “administered volume” may refer to either a blood transfusion or other any circulatory volume infusion, such as blood or blood products, crystalloid fluids, colloids, or bolus administration of a cardiovascular drug, or administration of other medications, fluids, or gasses. Furthermore, while reference may be made herein specifically to blood transfusions, such descriptions may apply to administration of any circulatory volume or therapeutic agents with a hemodynamic and/or cardiorespiratory pharmacological effect that requires titration. Additionally, while reference is made herein specifically to monitoring and controlling CV states, such descriptions may apply more generally to controlling and monitoring any circulatory state of a subject.

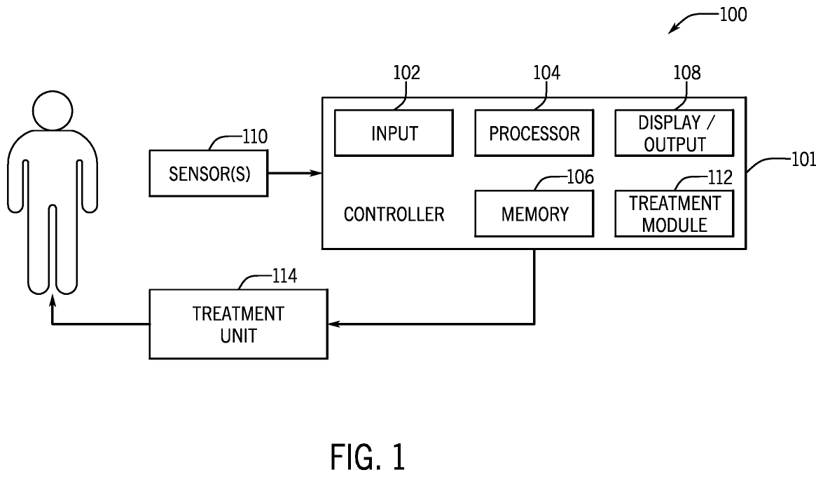

Referring particularly to FIG. 1, an example system 100 for monitoring and/or controlling a CV state of a subject during intravascular volume resuscitation is shown. In general, the system 100 may be any device, apparatus or system configured for carrying out instructions in accordance with the present disclosure. In some implementations, the system 100 may include a controller 101 with an input 102, a processor 104, a memory 106, and an output 108. The system 100 may also include one or more sensors 110 in communication with the controller 101 and configured to acquire CV data (e.g., physiological signals such as BP, heart rate (HR), continual tissue oxygenation, continual blood oxygenation, volumetric urine flow rate, and/or others) from a subject, intermittently or continuously. In some aspects, the sensors 110 may be configured to acquire data non-invasively from the subject. For example, one sensor 110 may include an attachable or wearable sensor, such as an oscillometric cuff. In other aspects, the sensor 110 may include an optical peripheral tissue oxygen saturation probe. In other aspects, the sensors 110 may be configured to acquire data from within a subject's anatomy, via insertable catheters or other interventional tools or devices. In some embodiments, as shown in FIG. 1, the system 100 and, more specifically, the controller 101 may also include a treatment module 112 configured to control a treatment unit 114, where the treatment unit 114 is configured to administer a fluid volume to the subject.

The system 100 may operate independently or as part of, or in collaboration with, a computer, system, device, machine, mainframe, or server. For example, in some aspects, the system 100, or the controller 101, may be portable, such as a mobile device, smartphone, tablet, laptop, or other portable device, apparatus or personal monitoring system. In this regard, the system 100 may be any system that is designed to integrate with a variety of software and hardware capabilities and functionalities, in accordance with the present disclosure, and may be capable of operating autonomously and/or with instructions from a user or other system or device. In such aspects, the controller 101 may be operably connectable to the treatment unit 114 to control delivery of a fluid volume or, alternatively, the controller 101 may act as a standalone monitoring system configured to provide automated decision support to a clinician. Furthermore, in some aspects, the system 100 may be a dedicated treatment system with an integrated treatment unit 114.

Generally, the input 102 may be configured to receive a variety of information from a user, a server, a database, and so forth, via a wired or wireless connection. The input 102 may include any number of input elements, for example, in the form of user interfaces such as touch screens, buttons, a keypad, a keyboard, a mouse, and the like, as well as compact discs, flash-drives or other computer-readable media. The input 102 may also include other types of input elements, such as a reader, a barcode scanner, an electronic scale, and the like. In some aspects, information provided via the input 102 may include information associated with the subject, such as subject characteristics, including age, weight, medical condition, and so forth, as well as certain CV data (e.g., CV parameters manually measured by a clinician rather than directly measured by connected sensor(s) 110, such as BP, heart rate (HR), continual tissue oxygenation, continual blood oxygenation, volumetric urine flow rate, and/or others), previous volume administration information, including volume, fluid type, and/or duration, and, optionally, selected administered pharmaceutical agent(s), dosing, and so forth. For example, any information associated with the subject's electronic medical record (EMR) may be retrieved via the input 102. In some implementations, information associated with a population may also be provided via input 102. In addition, a user may provide selections for time range of data analysis, or one or more desirable thresholds for particular CV parameters (such as, for example, high and low BP thresholds). In some aspects, reference data associated with previous measurements obtained from the subject or population may also be provided via input 102, or alternatively retrieved from the memory 106 or other storage location. Additionally, in some aspects, some or all information received via the input 102 may be stored in the memory 106, for example, via the processor 104.

Generally, the processor 104 may be configured to carry out steps for operating the system 100 using instructions stored in the memory 106. For example, the processor 104 may be configured to carry out steps for monitoring and controlling the CV state of a subject at one or more pre-determined time points by receiving and processing CV data, and other data or information obtained using sensors 110 or input 102, or both, either continuously or intermittently. In some aspects, the processor 104 is configured to determine a past time trajectory for one or more CV parameters, such as BP, and apply a statistical model, using the past time trajectory, volume administration, and other information, to determine a likelihood that the CV parameter will exceed or fall below a threshold at one or more pre-determined time points and/or a likelihood that an additional volume administration may be necessary.

More specifically, the processor 104 may be configured to apply a predictive model using CV data, previous volume administration information, and other information to determine a likelihood that the CV parameter will exceed or fall below a parameter at some future time point or range with and without an additional volume administration. For example, the processor 104 may analyze CV data, acquired at various time points or over a time range, to determine a present and estimated future CV state of the subject without additional volume administration. Using the estimated future CV state, the processor 104 may determine a likelihood that BP will be lower, or higher, than a predetermined threshold over a selected analysis period and/or for a certain duration. Furthermore, the processor 104 may estimate the future CV state of the subject in response to a new volume administration and determine a likelihood that BP will be lower, or higher, than the predetermined threshold.

In conducting a statistical analysis, the processor 104 may utilize a number of statistical techniques including Monte Carlo simulations, regression model analyses, and other formulae that provide a statistical result based on predicted future time trajectories, confidence intervals, volume administered, and other important clinical factors. In one aspect, as further described below, the processor 104 may utilize an autoregressive exogenous model. The processor 104 may determine confidence intervals for the determined future time trajectory of the CV parameter(s), for one or more points in time, by considering information from prior analyses, such as information obtained from population data, and/or previous observations from the subject.

As will be described, the processor 104 may communicate the estimated future CV states and likelihoods, which may be used to inform a clinician regarding a recommendation or potential necessity for additional volume to be administered to the subject. More specifically, the processor 104 may be configured to generate a report provided to a user or clinician via an output 108, for example, in the form of an audio and visual display or graphical user interface. The report may include a variety of information and data associated with the subject. For instance, the report may include an indication regarding a present CV state of the subject and/or estimated future CV state of the subject with and without additional volume administration. In some aspects, the report may display a past and/or future time trajectory for one or more CV parameters along with respective confidence intervals, and provide an alert or notification related to a likelihood that the one or more CV parameters will fall outside one or more thresholds at one or more pre-determined time points (e.g., with and without additional volume administration). In addition, the report may provide an indication or recommendation to a clinician regarding the timing of volume administration for maintaining or achieving a target CV state (such as maintaining a CV parameter within a pre-specified range). In other words, the generated report may provide decision support to the clinician during intravascular volume resuscitation.

In some implementations, the processor 104 may be configured to use the above estimates and likelihoods to determine and implement an optimized treatment for controlling the CV state of the subject. For example, the processor 104 may generate a report as a control output to the treatment module 112. In response, the treatment module 112 can control the treatment unit 114 to deliver the optimized treatment to a subject. As described, the condition of a subject may vary with time, and hence the processor 104 may be configured to determine an optimized treatment iteratively or periodically to account for changing clinical conditions (e.g., based on natural drift in a subject's CV parameters as well as additional volume administrations or other changes).

As noted above, in some aspects, the system 100 may be a dedicated treatment system with an integrated treatment unit 114. For example, in one aspect, the system 100 may be a blood transfusion machine, such as an infusion pump, elastomeric pump, or other suitable pump configured to administer blood to a subject. In this example, the controller 101 can be a pump controller, including a processor 104 and a memory 106; the treatment unit 114 can include at least a pump configured to control a flow of blood, for example, from a blood bag, to a subject (e.g., at a rate and duration as instructed by the pump controller); the input 102 can include a user interface having, for example, a touch screen, buttons, and/or dials; and the output 108 can include a display, such as an LCD display or other suitable display. In some aspects, the pump controller can also be configured to receive data from external sensors 110 and/or external inputs 102 (such as a barcode scanner, an electronic scale, or suitable external inputs). As described, the system 100, that is, a blood transfusion machine, may utilize computation results completed by the processor 104 to automatically and proactively deliver infusions, thus providing an improvement over current, manually controlled systems.

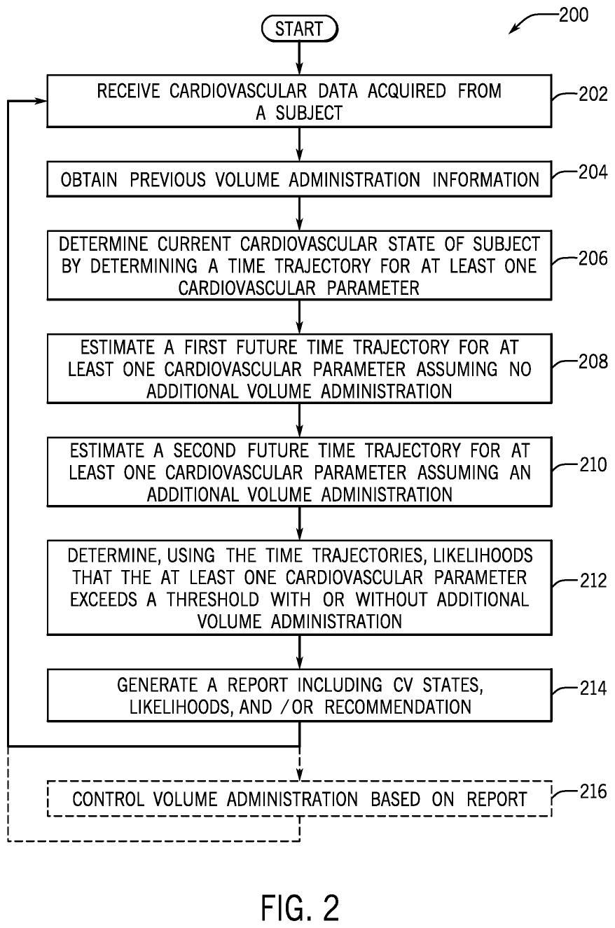

Turning now to FIG. 2, steps of a process 200 for monitoring and/or controlling a CV state of a subject are shown. Generally, the process 200 illustrates a novel use of a predictive model to provide automated decision support and/or intervention, for example, during intravascular volume resuscitation. More specifically, an autoregressive exogenous model may be used to forecast future BP using, as an exogenous input, volume administered over time. In some aspects, the process 200 may be implemented using a system, such as system 100 described with reference to FIG. 1, either continuously or intermittently. While reference will be made below to system 100 and its components, it should be noted that the process 200 may be carried out on other suitable systems, for example, with other components not specifically described herein.

Generally, the process 200 may begin at process block 202 where CV data is received or acquired. At process block 204, previous volume administration information is obtained. At process block 206, a current CV state of the subject is determined by analyzing the CV data, volume administration information, and/or other information. At process block 208, a first future time trajectory for at least one CV parameter, such as BP, is determined by analyzing the CV data and previous volume administration information based on an assumption that no additional volume will be administered. At process block 210, a second future time trajectory for the at least one CV parameter is determined by analyzing the CV data and previous volume administration information based on an assumption that an additional volume will be administered. At process block 212, using the forecasted future CV parameter and one or more thresholds, probabilities that the CV parameter will exceed or fall below the thresholds are determined. At process block 214, a report indicative of the future CV state of the subject and the determined probabilities may be generated.

More specifically, the process 200 may begin at process block 202 where CV data and, optionally, other physiological data associated with a subject may be received or acquired. For example, process block 202 may be implemented using system 100 using various sensors 110, such as BP sensors (e.g., an oscillometric cuff, an indwelling arterial catheter, or other suitable sensors), and/or other inputs 102 (e.g., data manually entered by a clinician, retrieved from external storage, etc.). In some aspects, such information can be automatically retrieved continuously or intermittently using sensors 110 (such as a continuous indwelling arterial catheter) or inputs 102. For example, an input 102 in the form of a strain gauge or electronic scale may be used to continuously or intermittently weigh a urine collection bag to acquire urine output data. Alternatively or additionally, intermittent prompts for such information may be utilized. As such, process block 202 may include a notification reminder (e.g., via the output 108) for a clinician to measure or input such information. For example, a reminder notification may be generated to prompt a clinician to acquire such information via a connected sensor 110 (such as with an oscillometric cuff connected to the system 100) or to acquire such information via an independent sensor and manually enter the information via the input 102.

In some aspects, intermittent data acquisition may be achieved at constant time intervals (e.g., every five minutes, every fifteen minutes, or another suitable interval). Additionally, intermittent data acquisition may be achieved at varying time intervals. For example, data acquisition times may be based on a projected future CV state. More specifically, data acquisition times may be based on a computed probability that a forecasted CV parameter will be different from the last acquired measurement (e.g., by a configurable threshold). In this manner, process block 202 may use feedback from other process steps below to set new data acquisition time intervals (e.g., when the CV parameter is predicted to change). In another example, data acquisition times may be based on previous CV data, such that a first configurable interval is used when a previously obtained CV parameter is within a first range or exceeding or falling below a first threshold (e.g., every five minutes when last obtained BP is considered “low”) and a second configurable interval is used when a previously obtained CV measurement is within a second range or exceeding or falling below a second threshold (e.g., every fifteen minutes when last obtained BP is considered “normal”).

Also, in some aspects, process block 202 may include retrieving information from memory 106 or another storage location. For example, a subject's reference data or other data associated with the subject may be previously stored in memory 106. Alternatively, a subject's information may be retrieved from the subject's EMR (e.g., via a wired or wireless connection to a server or database containing such information). Furthermore, certain information associated with a subject may be determined automatically using various measured quantities. For example, measured BP can be used to calculate mean arterial pressure (MAP) (as such, herein, any reference to BP may refer to BP or MAP, and vice versa). In addition, confidence intervals and other data obtained from a population of subjects, may also be retrieved or provided.

At process block 204, volume administration information is obtained. Volume administration information may include volume administered over time, time of administration, type of fluid administered (e.g., type of blood product, crystalloid fluid, colloid, drug, medication, other fluid, oxygen, etc.), and/or other information. Such information may be obtained via direct input by a user through the inputs 102, such as via buttons, touch screens or other input elements, via non-user inputs 102, such as an electronic scale, strain gauge, or barcode scanner, or may be obtained by communicating with various systems or devices, or treatment units, such as a separate programmable blood infusion system or device. For example, such information may be input at the time of administration, stored in memory 106, and then obtained from memory 106 when required at process block 204. Additionally, such information may be obtained from the subject's EMR. Furthermore, any information manually entered or obtained in process block 204 (and/or process block 202) may be stored and/or entered into the subject's EMR.

In some aspects, to input such information, a user may select from a variety of input options, for example, via input 102 (e.g., a touch screen, buttons, or other input element). According to one example, the user may select an option signifying that a new unit of blood is hung for infusion into the subject, and may further select a time option, including but not limited to: “infused in less than 15 minutes using a pressure bag,” “infused in 15-30 minutes using a pump,” “infused in 30-60 minutes using a pump,” or other input options. In this example, an estimated infused volume over time may be obtained (that is, computed by the processor 104) based on the inputs and a default “per unit” volume or a volume input by the user.

In another example, a system 100 may include an input 102 in the form of a barcode scanner. A user may scan the fluid to be infused near the time when infusion is initiated, such as a blood product labeled with a bar code containing information about product volume. From this input, an estimated infused volume over time may be obtained (that is, computed by the processor 104) using, for example, a default infusion time or an infusion time input by the user.

In yet another example, a system 100 may include an input 102 in the form of an electronic scale. A user may hang the fluid to be infused (such as a blood product in a bag) on a hook of the scale to weigh the bag over the duration of infusion. Based on the measured weight over time, an estimated infused volume over time may be obtained (that is, computed by the processor 104).

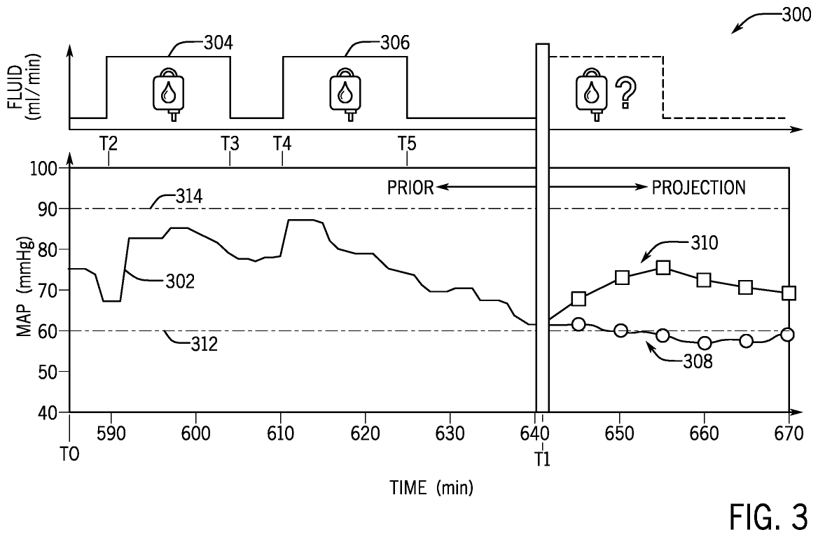

Referring now to process block 206, a current CV state of the subject may be determined, for example, using the CV data from process block 202 and the volume administration information (such as estimated volume over time) from process block 204. More specifically, at process block 206, a past time trajectory for at least one CV parameter may be determined by analyzing CV data and, optionally, volume administration information, and/or other information provided. For example, FIG. 3 illustrates a chart 300 of BP over time, including a past BP trajectory (line 302) from time T0 (585 minutes) to present time T1 (about 640 minutes). As shown in FIG. 3, volume administration information is also considered in chart 300, including a first volume administration 304 from T2 (590 minutes) to T3 (605 minutes) and a second volume administration 306 from T4 (610 minutes) to T5 (625 minutes).

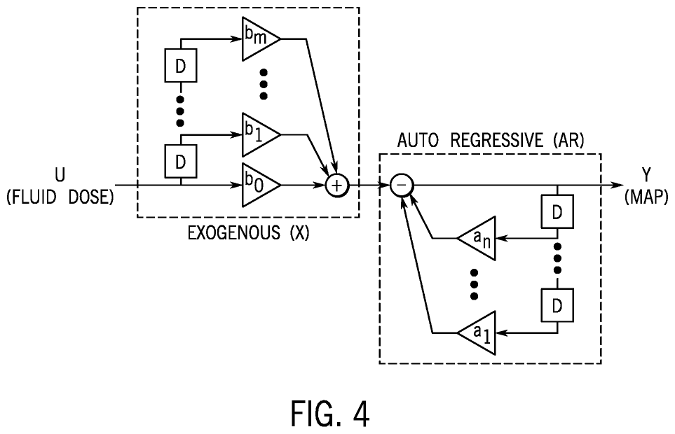

At process block 208, a first statistical analysis may be performed to determine a future CV state of the subject (that is, after time T1) using the CV data and volume administration information and assuming no additional volume is to be administered over a predetermined time period after time T1. In some aspects, the statistical analysis uses an autoregressive exogenous (ARX) model, where CV parameters are endogenous variables in the model, while the volume administration information (such as volume transfused over time) is the exogenous input to the model. For example, the ARX model may be used by the processor 104 to forecast a future CV parameter, such as BP, based on prior BP trend data and prior volume administration data. An example ARX model is illustrated in FIG. 4 and mathematically shown below, where D (in FIG. 4) is a delay or backward shift by one sampling time, Y is the output CV parameter (such as BP or MAP), U is the input volume administration information, aₖ is the autoregressive coefficient, and bₖ is the exogenous coefficient:

$$y ( n ) = - {\sum }_{k = 1}^{n} {a}_{k} y ( n - k ) + {\sum }_{k = 0}^{m} {b}_{k} u ( n - k )$$

FIG. 3 also shows an example output from the ARX model used in process block 208 in the form of a first future time trajectory (line 308). As shown in FIG. 3, the future time trajectory 308 illustrates a drop in BP after time T1.

Additionally, at process block 210, a second statistical analysis may be performed to determine a future CV state of the subject using CV data and volume administration information and assuming an additional volume is to be administered over a predetermined time period after time T1. This predetermined time period may start at time T1, or may start at some delayed time after T1 (such as, for example, five minutes). In some aspects, the predetermined time period and/or the delayed time can be configurable parameters (e.g., input by a user via inputs 102). Similar to process block 208, process block 210 may implement an ARX model, as described above, to output future CV state assuming the additional volume input. FIG. 3 shows an example output from the ARX model used in process block 210 in the form of a second future time trajectory (line 310). As shown in FIG. 3, the future time trajectory 310 illustrates an increase in BP after time T1.

In some aspects, process block 210 may include more than one statistical analysis based on multiple different future contingencies. In other words, additional statistical analyses may be performed to determine multiple future CV states of the subject assuming different future administrations (e.g., a first analysis assuming a first volume of “x” amount, a second analysis assuming a second volume of “y” amount, etc. or a first analysis assuming a volume administered at time “x” after T1, a second analysis assuming a volume administered at time “y” after T1, etc.).

The above statistical models may be used to determine probabilities or likelihoods that one or more CV parameters, such as BP, exceeds or falls below predetermined thresholds or ranges of values at one or more predetermined time points or within a predetermined time range after T1. As such, at process block 212, using the forecasted future CV states from process blocks 208 and 210 and one or more thresholds, probabilities that the forecasted CV parameter will exceed or fall below the thresholds are determined.

For example, process block 212 may include determining a probability that BP will exceed or fall below a threshold if no volume is administered and another probability that BP will exceed or fall below a threshold if volume is administered (and/or additional probabilities if additional future contingencies are considered, as described above). More specifically, process block 212 may use a low threshold and/or a high threshold. Such thresholds may be preset defaults and/or may be configurable by a user (e.g., via the input 102). As shown in FIG. 3, a low threshold may be set at 60 mmHg (line 312) and a high threshold may be set at 90 mmHg (line 314). Notably, the forecasted BP with volume administration (line 310) remains between the thresholds after time T1, while the forecasted BP without volume administration (line 308) falls below the low threshold about ten minutes after time T1. Thus, given certain confidence intervals and/or other factors, a probability of future BP below the low threshold without another volume administration may be substantially high, whereas a probability of future BP below the low threshold with another volume administration may be substantially low. Probabilities may be determined as specific percentages or non-number values, such as high, low, intermediate, etc. Furthermore, in aspects where there is a low threshold and a high threshold, a goal range may be set (that is, a range between the low threshold and the high threshold), and process block 212 may include determining a probability that BP will remain within the goal range.

The determination at process block 212 may be made with respect to particular points in time after T1, such as five, ten, fifteen, twenty minutes, and/or another time after T1. In some aspects, the points in time may be configured as any times after T1, any times after T1 between one and 60 minutes, or another suitable time range after T1. The determination may also be made for a single point in time or multiple discrete times (for example, FIG. 3 illustrates charted values at discrete time points every five minutes after T1). Additionally, the determination may be made with respect to specified durations of time. More specifically, the determination at process block 212 can further identify probabilities that the subject's BP will exceed or fall below a threshold for a set duration of time (e.g., two minutes or another set number) or for a number of consecutive discrete time points.

Additionally, in some aspects, process block 212 may include a further determination that combines the above-described probabilities from the two or more future forecasts into a likelihood of an additional administered volume being needed. For example, if there is a high probability of exceeding or falling below thresholds when no volume is administered, and a low probability of exceeding or falling below thresholds when volume is administered, a determination may be made of a high likelihood that volume administration is needed. However, in some situations, it may be possible that BP is projected to drop below the low threshold if no volume is administered, but BP is also projected to rise above the high threshold if volume is administered. For example, a probability of low BP below the low threshold without volume administration may be 18%, while a probability of high BP above the high threshold with volume administration may be 42%. Accordingly, process block 212 may consider the above probabilities with respect to set probability thresholds to determine the likelihood of needing volume administered. Alternatively or additionally, process block 212 may include performing a cost function analysis that quantifies a priority of preventing the CV parameter from exceeding or falling below one threshold if no volume is administered versus preventing the CV parameter from exceeding or falling below another threshold if volume is administered based on the determined probabilities.

The probabilities or likelihoods determined at process block 212 may inform a determination for a new volume administration, that is, to maintain the CV parameter within or between the thresholds. For example, at process block 214, a report indicative of the future CV states of the subject and determined probabilities may be generated. The report may be in any form, and include any information. In some aspects, the report may be in the form of an audio or visual notification provided to a clinician, such as through a graphical user interface (e.g., an output 108 of system 100). In some aspects, a separate notification may be provided via a remote communication, such as via a text message or central display station. Based on the report and/or notification, a clinician may choose to administer an additional volume (e.g., via gravity, a pressure bag, an infusion pump, or another suitable technique).

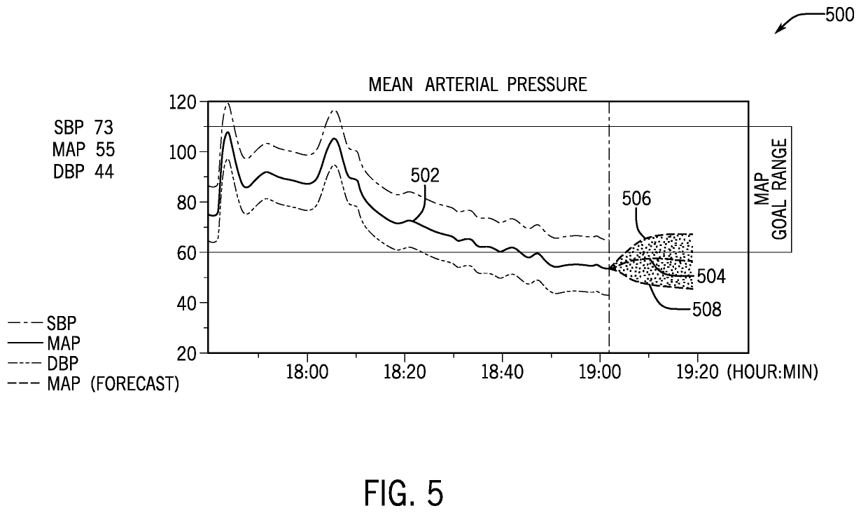

For instance, the generated report may indicate that particular CV parameters exceed or fall below selected ranges or thresholds in the current CV state and/or probabilities related to future CV states, such as an imminent or sustained risk for falling below the low threshold (e.g., hypotension), exceeding the high threshold (e.g., hypertension), and so forth. By way of example, one report may include time traces for BP with two future projections (with and without volume administration), each identifying probabilities of imminent hypertension or hypotension over future time points, e.g., similar to the chart 500 illustrated in FIG. 5. More specifically, FIG. 5 illustrates an example chart 500 including a past BP trajectory 502 and an estimated future BP trajectory 504 including a confidence interval (e.g., in the shape of a cone, having an upper limit 506 and a lower limit 508). While the chart 500 of FIG. 5 illustrates a single future BP trajectory 504, it is contemplated herein that generated reports may include one, two, three, or more future trajectories.

According to another example, generating a report may include generating a recommendation for volume administration. More specifically, a report may include a likelihood or recommendation that a clinician initiate volume administration based on the above determinations at process block 212. For instance, the report may include a recommendation to initiate based on a high likelihood of dropping below a low threshold without volume administration, or a recommendation not to initiate based on a high likelihood of exceeding a high threshold with volume administration. Such recommendations may be based on the determined probabilities or likelihoods exceeding or falling below a specific value and/or the above-described cost function analysis. In some aspects, additional notifications may be further provided only when such probabilities indicate intervention is recommended (e.g., when the projected probability of dropping below the threshold without volume administration exceeds a set number). Additionally, in some aspects, when multiple future contingencies are analyzed (e.g., different future volume amounts, durations, etc.), the recommendation may further include a recommended volume administration option (e.g., based on a determined optimal future contingency). Accordingly, in such aspects, rather than a “yes” or “no” recommendation to administer volume, the recommendation may provide additional guidance regarding amount and/or duration of the volume administration.

Thus, in some aspects, the report may provide an indication to a clinician for administering additional volume. Accordingly, the process 200 may provide automated decision support for controlling BP between two thresholds to, e.g., avoid risk of worsening volume loss (if exceeding the high threshold), thereby necessitating additional volume administration or reduce risk of ischemia in essential organs (if falling below the low threshold). As may be appreciated from the above description, this approach may be fully configurable. For instance, in managing BP, precise control may be achieved by selecting a narrow range for allowable BP values and narrow time intervals, while permissive control would imply wider goal range and time intervals, with tolerance for brief excursions outside the range. Thus, when wider ranges are set, a number of clinician notifications may be minimized, resulting in fewer interruptions to clinician in the typical clinical setting.

Alternatively, or additionally, process block 214 may include generating a report as an output for controlling a treatment unit to automatically administer a fluid volume at process block 216. In other words, the estimated future CV states and probabilities may be used to identify whether action should be taken by a system. For example, when system 100, such as a blood transfusion machine, carries out the present method steps, the report may serve as a control output for the treatment unit 114 to initiate volume administration at a set time and/or for a set duration based on the above determinations. In other words, rather than generating an audio or visual notification that a volume administration is recommended, an output is generated to directly initiate volume administration. Furthermore, when a system automatically administers a volume, a record of the new volume administration may be generated and saved, for example, in system memory and/or the subject's EMR.

Accordingly, process block 214 may generate a report for controlling the CV state of the subject at one or more time points, either by a clinician or a system configured to do so. Furthermore, process blocks 202-214 (or process blocks 202-216) may be repeated in a loop so that new reports are periodically generated based on updated information (that is, new CV data and new volume administration information), thus providing new cardiovascular states, likelihoods, and/or recommendations over time. As a result, substantially constant decision support and/or control may be provided during intravascular volume resuscitation to indicate when additional fluid volume should or should not be administered. This can facilitate treatment in today's busy medical settings, particularly for patients who are hemorrhaging internally or externally due to trauma, surgery, etc.

Furthermore, a blood transfusion machine operating in accordance with process 200 provides an improvement over current, manually controlled transfusion machines by utilizing computation results to continuously control and better deliver transfusions during intravascular volume resuscitation. In other words, a blood transfusion machine implementing process blocks 202-216 can form a closed-loop system for controlling the CV state of a subject. Accordingly, the blood transfusion machine implementing the above process 200 may achieve intravascular volume resuscitation in a proactive manner by predicting future CV states and acting accordingly, in comparison to current methods, where clinicians implement such resuscitation in a reactive manner by only relying current CV parameters.

In summary, the present disclosure provides systems and methods for monitoring and/or controlling a CV states of a subject during intravascular volume resuscitation. In some aspects, present and potential future CV state of a subject are predicted in order to provide automated decision support and inform volume administration by a clinician or a system suitable to do so.

As described above, future CV states may be predicted using an ARX model that incorporates previous volume administrations as an exogenous input. The ARX model is applicable to intravascular volume resuscitation because the clinical intervention is administered in a rapid and intermittent manner. Accordingly, the methods described herein may be generally applicable to any intervention that has a relatively rapid and observable effect on blood pressure, such as blood transfusions, as discussed above, as well as bolus administration of crystalloid fluid, bolus administration of a cardiovascular drug, or other administrations.

For example, while the above aspects are generally described with respect to predicting BP using blood transfusion as an input, the systems and methods described herein may be used to predict future BP using measure inputs or outputs of fluids, blood, blood pressure reduction medication (e.g., as a bolus administration or slow infusion), vasopressor medication, urine output, heart rate, and/or tissue oxygenation saturation. For example, while the above description generally discusses fluid or blood volume administration to maintain BP above a low threshold, these methods and systems may apply to administration of BP reduction medication to maintain BP below a high threshold (e.g., for subjects who are hypertensive). Additionally, while some of the above inputs may not be periodically administered by a clinician, they still contain input/output information applicable to BP monitoring and predictions. For example, observing a decreasing urine output would suggest that future BP will drop.

In another example, the systems and methods described herein may be used to predict and/or control future urine output using exogenous variables such as fluids, blood, and/or BP. In some aspects, this example may be applicable to subjects suffering large burns, whom require fluid resuscitation to reduce dehydration risk. According to this example, urine output and previous fluid administration may be considered to estimate a future urine output trajectory, and a report may be generated that guides future fluid administration so that the likelihood that urine output falls outside a threshold is minimized.

In yet another example, the systems and methods may be used to predict and/or control future HR using exogenous variables such as HR control medication (e.g., nodal blockage agents used for treating atrial fibrillation with rapid ventricular response) or sedatives used, for example, to treat alcohol withdrawal (e.g., valium, Ativan, or another suitable medication). In this example, HR may be monitored and future predictions determined to recommend administering or not administering an HR reduction medication to keep HR within a goal range (e.g., for subjects who are too tachycardic). Alternatively, HR may be monitored and future predictions determined to recommend administering or not administering a sedative in order to maintain HR between upper and lower thresholds (e.g., between 50 beats per minute (BPM) and 100 BPM).

In another example, the systems and methods may be used to predict and/or control future oxygen saturation percentage using exogenous variables such as inspired oxygen flow rate. This example may be used to help prevent hypoxia while minimizing oxygen toxicity risk. In each of these examples, the systems and methods herein may directly control or help a clinician control new interventions in a proactive manner using predictive modeling.

Furthermore, notably, the ARX model and rapid or intermittent infusion applications described herein are in contrast to previously described autoregressive (AR) models for slow or constant interventions, such as long-term drug infusions that impact blood pressure. For example, if an intervention is applied slowly, blood pressure changes gradually over time, making it mathematically difficult to determine the effect of the intervention versus the underlying drift in BP (i.e., a natural change over time that is unrelated to the intervention). As such, an AR model considers the system as a whole to predict a future state assuming the system will continue operating as it has in the past. Thus, an AR model may be used to provide an estimated future trajectory for a parameter given no changes to a system, but because such a model cannot consider how an intervention independently affects the parameter, the AR model cannot provide predicted trajectories based on a new intervention being administered.

An ARX model, on the other hand, considers how an intervention independently affects a parameter and can therefore provide predicted trajectories based on a new intervention being administered. For example, when an intervention is applied very quickly or intermittently, it is possible to mathematically estimate the natural drift in the BP that appears before and during the intervention and, based on any rapid change in BP that is simultaneous with the intervention, independently estimate the effect of the intervention. This estimation can become even more accurate if the effect of the intervention on BP is analyzed based on a plurality of interventions (e.g., if the intervention is applied several times, such as the two previous volume administrations illustrated in FIG. 3). The ARX model described herein, unlike an AR model, can account for changes to the system created by this independent effect and can therefore better predict future CV states for rapid or intermittent infusion applications.

Furthermore, while an AR model may be applicable for slow infusion applications (because infusion dose is changed infrequently), but not applicable to rapid infusion applications, the ARX model may be applicable to rapid as well as slow infusion applications. For example, as noted above, it may be difficult to determine how an intervention affects a subject's individual system as an exogenous variable in slow infusion applications, given the substantially constant infusion rate. However, population data may be used to estimate this effect. In other words, population data (e.g., illustrating a system showing variability caused by an intervention) as well as the subject's data (e.g., illustrating the system with constant intervention and minimal variation) may be considered to estimate an independent effect of the intervention that can be utilized by an ARX model.

The present invention has been described in terms of one or more embodiments, including preferred embodiments, and it should be appreciated that many equivalents, alternatives, variations, and modifications, aside from those expressly stated, are possible and within the scope of the invention. As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

1. A system for monitoring a state of a subject, the system comprising:
a sensor configured to acquire cardiovascular data from the subject;
a processor configured to:
obtain previous volume administration information,
analyze the cardiovascular data to determine a past time trajectory for at least one cardiovascular parameter,
determine, using the past time trajectory and the previous volume administration information, a future state of the subject with and without additional volume administration,
determine, based on the future state of the subject, a likelihood that the at least one cardiovascular parameter will exceed or fall below a threshold at one or more pre-determined time points, and
generate a report indicative of the future state of the subject; and
an output configured to display the report to a user.

2. The system of claim 1, wherein the sensor is one of an oscillometric cuff and an indwelling arterial catheter.

3. The system of claim 1, wherein the output is an LCD display.

4. The system of claim 1, wherein the processor is further configured to make a recommendation for administering an additional volume to the subject based on the determined likelihood, and generate the report including the recommendation.

5. The system of claim 4, wherein the processor is further configured to control the future state of the subject based on the recommendation.

6. A system for controlling a state of a subject, the system comprising:
a treatment unit configured to administer a fluid volume to the subject; and
a processor configured to:
receive cardiovascular data acquired from the subject,
obtain previous volume administration information,
analyze the cardiovascular data to determine a past time trajectory for at least one cardiovascular parameter,
determine, using the past time trajectory and the previous volume administration information, a future state of the subject with and without additional volume administration,
determine, based on the future state of the subject, a likelihood that the at least one cardiovascular parameter will exceed or fall below a threshold at one or more pre-determined time points,
generate a report including a recommendation for administering an additional volume to the subject based on the determined likelihood, and
control the treatment unit to administer the fluid volume to the subject based on the report.

7. The system of claim 6 and further comprising a sensor configured to acquire the cardiovascular data from a subject.

8. The system of claim 6, wherein the treatment unit is a pump.

9. The system of claim 6, wherein the processor is configured to determine the future state of the subject using a statistical model.

10. The system of claim 9, wherein the statistical model is an autoregressive exogenous model, and the processor is configured to use the previous volume administration information as an exogenous input to the autoregressive exogenous model.

11. The system of claim 6 and further comprising an input configured to receive, from a user, the cardiovascular data acquired from the subject.

12. The system of claim 6, wherein the previous volume administration information includes administered volume over time.

13. The system of claim 6 and further comprising an input configured to receive the previous volume administration information.

14. The system of claim 13, wherein the input includes a user interface.

15. The system of claim 13, wherein the input includes one of an electronic scale and a barcode scanner.

16. A method for monitoring a state of a subject, the method comprising:
acquiring cardiovascular data from a subject;
obtaining previous volume administration information;
analyzing the cardiovascular data to determine a past time trajectory for at least one cardiovascular parameter;
determining a future state of the subject with and without additional volume administration using a statistical model with the cardiovascular data and the previous volume administration information as inputs to the statistical model;
determining, based on the future state of the subject, a likelihood that the at least one cardiovascular parameter will exceed or fall below a threshold at one or more pre-determined time points;
generating a report indicative of the future state of the subject; and
displaying the report to a user.

17. The method of claim 16, wherein the method further comprises making a recommendation for administering an additional volume to the subject based on the determined likelihood, and including the recommendation in the generated report.

18. A method for controlling a state of a subject, the method comprising:
acquiring cardiovascular data from a subject;
obtaining previous volume administration information;
analyzing the cardiovascular data to determine a past time trajectory for at least one cardiovascular parameter;
determining a future state of the subject with and without additional volume administration using a statistical model with the cardiovascular data and the previous volume administration information as inputs to the statistical model;
determining, based on the future state of the subject, a likelihood that the at least one cardiovascular parameter will exceed or fall below a threshold at one or more pre-determined time points;
generating a report including a recommendation for administering an additional volume to the subject based on the determined likelihood; and
controlling administration of an additional fluid volume to the subject based on the report.

19. The method of claim 18, wherein controlling administrating of the additional fluid volume includes controlling an infusion pump to administer the additional fluid volume to the subject.

20. The method of claim 18, wherein the statistical model is an autoregressive exogenous model, and the previous volume administration information is used as an exogenous input to the autoregressive exogenous model.

21. The method of claim 20, wherein the method further comprises periodically updating the future state of the subject based on new cardiovascular data and new volume administration information, and determining a new likelihood based on the updated future state.

22. The method of claim 18, wherein the at least one cardiovascular parameter includes one of blood pressure, heart rate, urine output, and tissue oxygen saturation.
